(19) United States
(12) Patent Application Publication
(10) Pub. No.: US 2014/0148711 A1
(43) Pub. Date: May 29, 2014

(54) Recursive Least Squares Adaptive Acoustic Signal Filtering for Physiological Monitoring System

(71) Applicants: Te-Chung Isaac YANG, Aliso Viejo, CA (US); Yongji FU, Cary, NC (US)

(72) Inventors: Te-Chung Isaac YANG, Aliso Viejo, CA (US); Yongji FU, Cary, NC (US)

(21) Appl. No.: 13/684,808

(22) Filed: Nov. 26, 2012

(51) Int. Cl. A61B 5/0205; A61B 5/00; A61B 7/00

(52) U.S. Cl. CPC A61B 5/0205; A61B 7/003; A61B 5/725; A61B 5/7278; A61B 5/6801
USPC 600/484

(57) ABSTRACT

Recursive least squares (RLS) adaptive acoustic signal filtering for a physiological monitoring system reduces residual heart sound in a primary signal remaining after application of a respiration sound bandpass filter to a first instance of a mixed signal containing respiration sound and heart sound. Residual heart sound in the primary signal is reduced by minimizing a component in the primary signal that correlates with a reference signal containing heart sound but almost no residual respiration sound after application of a heart sound bandpass filter to a second instance of the mixed signal. The correlative component in the primary signal is minimized by applying an adaptive filter to the reference signal and subtracting the filtered reference signal from the primary signal to produce a residue signal, wherein the coefficients for the adaptive filter are selected to minimize the least square error of the residue signal.

### BACKGROUND OF THE INVENTION

The present invention relates to physiological monitoring and, more particularly, filtering of an acoustic physiological signal containing respiration sound and heart sound to isolate respiration sound.

In acoustic physiological monitoring, estimates of physiological parameters, such as respiration rate and heart rate, are computed by analyzing an acoustic physiological signal captured by one or more sound transducers placed on the human body.

In ambulatory acoustic physiological monitoring, where a patient wears a physiological monitoring device as the patient goes about his or her daily routine, patient comfort and battery life impose significant restrictions on the size, weight and complexity of the monitoring device that require economical design. One way that design economy can be achieved is by using a single sound transducer to record a mixed signal containing both respiration sound and heart sound.

Before physiological parameters can be estimated from a mixed signal containing both respiration sound and heart sound, however, the respiration sound and heart sound must be disambiguated to enable them to be recovered. One way to disambiguate respiration sound and heart sound is to split the mixed signal into two parallel signals and apply to the parallel signals bandpass filters having passbands in the frequency domain of respiration sound and heart sound, respectively. For example, a respiration sound bandpass filter having a passband between 80 Hz and 300 Hz may be applied to one of the parallel signals to isolate respiration sound and a heart sound bandpass filter having a passband from 10 Hz to 100 Hz may be applied to the other parallel signal to isolate heart sound.

Unfortunately, applying a respiration sound bandpass filter to a mixed signal at best provides partial isolation of respiration sound. Heart sound often spreads well into the frequency domain for respiration sound. While heart sound is typically heard between 10 Hz and 100 Hz, some heart sound can be heard as high as 150 Hz. Moreover, because heart sound is typically much stronger than respiration sound, even a small amount of heart sound spread into the frequency domain for respiration sound can mask respiration events and lead to erroneous respiration parameter estimation, and can even prevent recovery of respiration sound altogether.

One way the heart sound frequency spreading problem might be eliminated is by raising the low cutoff frequency of the respiration sound bandpass filter above 80 Hz; however, this can inadvertently remove respiration sound and cause failure or error in estimating respiration parameters.

### SUMMARY OF THE INVENTION

The present invention, in a basic feature, provides recursive least squares (RLS) adaptive acoustic signal filtering for a physiological monitoring system.

The invention reduces residual heart sound in a primary signal remaining after application of a respiration sound bandpass filter to a first instance of a mixed signal containing respiration sound and heart sound. Residual heart sound in the primary signal is reduced by minimizing a component in the primary signal that correlates with a reference signal containing heart sound but almost no residual respiration sound after application of a heart sound bandpass filter to a second instance of the mixed signal. The correlative component in the primary signal is minimized by applying an adaptive filter to the reference signal and subtracting the filtered reference signal from the primary signal to produce a residue signal, wherein the coefficients for the adaptive filter are selected to minimize the least square error of the residue signal.

In one aspect of the invention, a recursive least squares (RLS) adaptive acoustic physiological signal filtering method, comprises the steps of capturing by a physiological monitoring system a mixed acoustic physiological signal containing respiration sound and heart sound; producing by the system a primary signal at least in part by applying a respiration sound bandpass filter to a first instance of the mixed signal; producing by the system a reference signal at least in part by applying a heart sound bandpass filter to a second instance of the mixed signal; producing by the system a filtered reference signal at least in part by applying an adaptive filter to the reference signal; producing by the system a residue signal at least in part by subtracting the filtered reference signal from the primary signal; computing by the system one or more values for one or more respiration parameters using the residue signal; outputting by the system respiration information based at least in part on the respiration parameter values; computing by the system one or more values for one or more coefficients for the adaptive filter in accordance with an RLS algorithm using the residue signal; and updating by the system the adaptive filter with the coefficient values.

In some embodiments, the primary signal is further produced by computing an energy envelope of the first instance of the mixed signal.

In some embodiments, the primary signal is further produced by downsampling the first instance of the mixed signal.

In some embodiments, the reference signal is further produced by computing an energy envelope of the second instance of the mixed signal.

In some embodiments, the reference signal is further produced by downsampling the second instance of the mixed signal.

In some embodiments, the respiration sound bandpass filter and the heart sound bandpass filter have respective passbands that partially overlap.

In some embodiments, the respiration sound bandpass filter has a passband from 80 Hz plus or minus ten percent to 300 Hz plus or minus ten percent.

In some embodiments, the heart sound bandpass filter has a high cutoff frequency from 10 Hz plus or minus ten percent to 100 Hz plus or minus ten percent.

In some embodiments, the method further comprises the step of splitting by the system the mixed signal into the first instance and the second instance.

In some embodiments, the method further comprises the step of amplifying by the system the mixed signal.

In some embodiments, the method further comprises the step of applying by the system a lowpass filter to the mixed signal.

In some embodiments, the respiration parameters include respiration rate.

In some embodiments, the system is an ambulatory monitoring system.

In another aspect of the invention a physiological monitoring system comprises a sound capture system configured to capture a mixed acoustic physiological signal containing respiration sound and heart sound; an acoustic signal processing system operatively coupled with the capture system and configured to produce a primary signal at least in part by applying a respiration sound bandpass filter to a first instance of the mixed signal, produce a reference signal at least in part by applying a heart sound bandpass filter to a second instance of the mixed signal, produce a filtered reference signal at least in part by applying an adaptive filter to the reference signal, produce a residue signal at least in part by subtracting the filtered reference signal from the primary signal, compute one or more values for one or more respiration parameters using the residue signal, output the respiration parameter values, compute one or more values for one or more coefficients for the adaptive filter in accordance with an RLS algorithm using the residue signal and update the adaptive filter with the coefficient values; and a physiological data output system operatively coupled with the processing system and configured to output respiration information based at least in part on the respiration parameter values.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a physiological monitoring system in some embodiments of the invention.

FIG. 2 shows an acoustic signal processing system in some embodiments of the invention.

FIG. 3 shows an RLS adaptive filtering unit in some embodiments of the invention.

FIG. 4 shows an RLS adaptive acoustic signal filtering method in some embodiments of the invention.

### DETAILED DESCRIPTION OF A PREFERRED EMBODIMENT

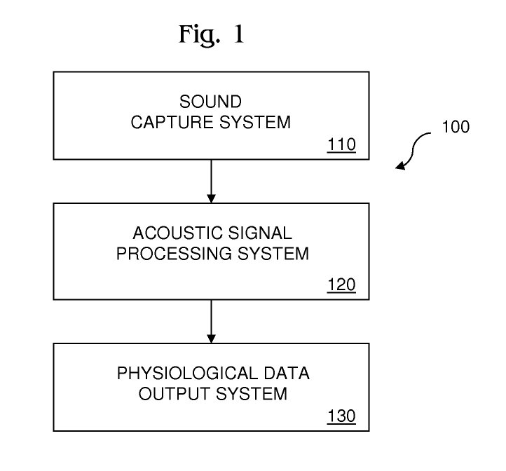

FIG. 1 shows a physiological monitoring system 100 in some embodiments of the invention. Monitoring system 100 includes a sound capture system 110, an acoustic signal processing system 120 and a physiological data output system 130, which are communicatively coupled in series.

Capture system 110 includes a sound transducer that detects body sound, including respiration sound and heart sound, at a detection point, such as the trachea, chest or back of a person being monitored, and continually transmits a mixed acoustic signal containing the detected body sound to processing system 120. Capture system 110 may include, for example, a microphone positioned on the body of a human subject that detects the body sound. Capture system 110 also includes an amplifier, a lowpass filter and an analog/digital (A/D) converter that transform the detected body sound into the mixed signal. Detected body sounds are represented in the mixed signal as a time sequence of digital samples of various amplitudes.

Processing system 120, under control of a processor executing software instructions, receives the mixed signal from capture system 110, generates values for one or more respiration parameters for the person being monitored during different time segments of the mixed signal and transmits the values to output system 130. In some embodiments, monitored respiration parameters include respiration rate, fractional inspiration time and/or inspiration to expiration time ratio (I:E). Processing system 120 may additionally generate and transmit to output system 130 values for other physiological parameters, such as heart rate.

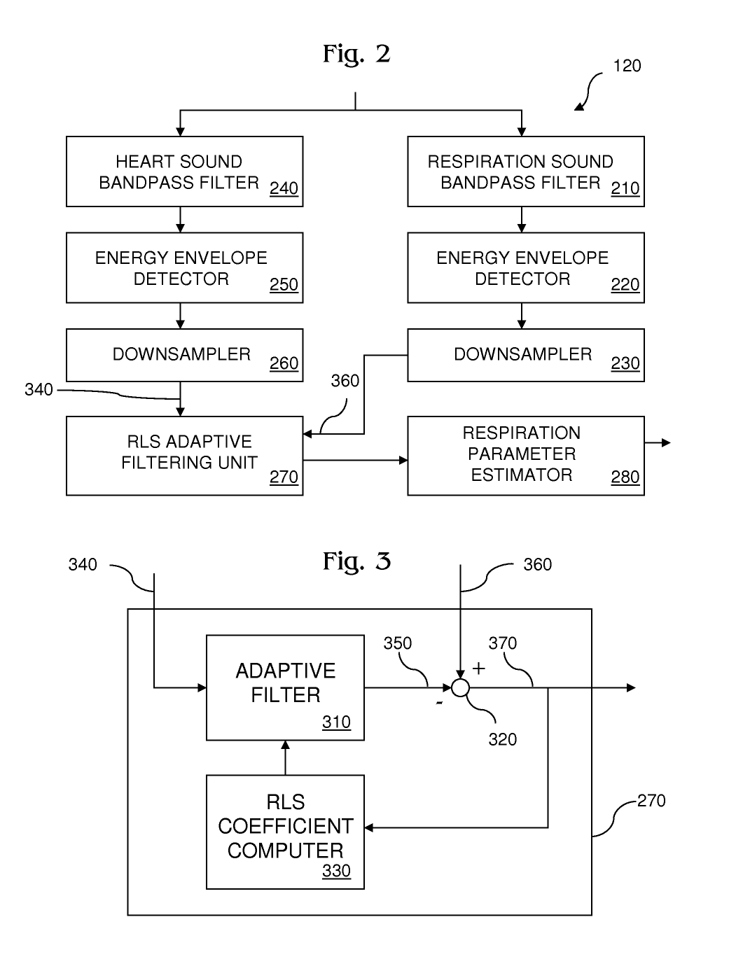

FIG. 2 shows processing system 120 in some embodiments of the invention. When processing system 120 first receives the mixed signal from capture system 110, respiration sound and heart sound are intermingled so as to be unrecoverable. Processing system 120 splits the mixed signal into a first instance and second instance that processing system 120 processes on parallel paths to produce a primary signal and a reference signal, respectively.

On one parallel path, processing system 120 applies a respiration sound bandpass filter 210 to the first instance of the mixed signal. Filter 210 has a passband in the frequency domain of respiration sound. In some embodiments, filter 210 has a passband from 80 Hz to 300 Hz, although in other embodiments the low cutoff frequency may vary plus or minus ten percent from 80 Hz and the high cutoff frequency may vary plus or minus ten percent from 300 Hz. After application of filter 210, an energy envelope detector 220 computes an energy envelope of the first instance of the mixed signal after which downsampler 230 downsamples the energy envelope to produce a primary signal 360 supplied as an input to RLS adaptive filtering unit 270. In some embodiments, each data point of the energy envelope is computed as the variance of the first instance of the mixed signal over a small group of consecutive data samples, which is representative of the total energy of the signal during a short time window, and consecutive data points of the energy envelope are computed from consecutive non-overlapping small groups of data samples of the same size. It bears noting that the loudness of sounds is generally proportional to the amplitude of data points in the energy envelope. Thus, troughs in the energy envelope represent quiet times and peaks or spikes in the energy envelope represent loud times. In other embodiments, the energy envelope may be computed using a Hilbert transform. After computation of the energy envelope, downsampler 230 downsamples the energy envelope to a lower sampling rate to produce primary signal 360, which is supplied as an input to RLS adaptive filtering unit 270. In other embodiments, downsampling may be integrated with energy envelope detection by, for example, computing the energy envelope from non-consecutive time windows (i.e., “skipping” time windows in energy envelope computation).

On the other parallel path, processing system 120 applies a heart sound bandpass filter 240 to the second instance of the mixed signal. Filter 240 has a passband in the frequency domain of heart sound. In some embodiments, filter 240 has a passband from 10 Hz to 100 Hz, although in other embodiments the low cutoff frequency may vary plus or minus ten percent from 10 Hz and the high cutoff frequency may vary plus or minus ten percent from 100 Hz. After application of filter 240, an energy envelope detector 250 computes an energy envelope of the second instance of the mixed signal after which downsampler 260 downsamples the energy envelope to produce a reference signal 340 supplied as an input to RLS adaptive filtering unit 270. Energy envelope computation and downsampling of the second instance of the mixed signal are performed in generally the same manner as energy envelope computation and downsampling of the first instance of the mixed signal.

Due to heart sound spreading into the frequency domain for respiration sound and the strength of heart sound relative to respiration sound, primary signal 360 contains both respiration sound and a meaningful level of residual heart sound. On the other hand, due to the relative weakness of respiration sound, reference signal 340 contains heart sound but virtually no residual respiration sound. Accordingly, RLS adaptive filtering unit 270 reduces the residual heart sound in primary signal 360 by applying adaptive filtering in accordance with a rule of least square error to reduce a component in primary signal 360 that correlates with reference signal 340.

FIG. 3 shows RLS adaptive filtering unit 270 in some embodiments of the invention. An adaptive filter 310 receives as an input reference signal 340 resulting from application of heart sound bandpass filter 240 (as well as energy envelope detector 250 and downsampler 260) to a mixed signal containing both respiration sound and heart sound. Due to application of filter 240, reference signal 340 contains heart sound but almost no residual respiration sound. Filter 310 produces as an output a filtered reference signal 350 which is supplied as one input to subtractor 320. Subtractor 320 receives as another input primary signal 360 resulting from application of respiration sound bandpass filter 210 (as well as energy envelope detector 220 and downsampler 230) to the mixed signal. After application of filter 210, primary signal 360 contains both respiration sound and a meaningful level of residual heart sound. Subtractor 320 subtracts filtered reference signal 350 from primary signal 360 to produce a residue signal 370. Residue signal 370 is supplied as feedback to an RLS coefficient computer 330, which uses residue signal 370 to compute new values for one or more coefficients of filter 310 in accordance with an RLS algorithm. By way of example, coefficient computer 330 may compute new coefficient values wₙ for filter 310 designed to minimize a weighted least square error cost function C(wₙ) that is related to residual signal 370 e(i) according to

$$C ( {w}_{n} ) = {\sum }_{i = 0}^{n} {λ}^{n - i} {e}^{2} ( i )$$

where n is a tap size of filter 310 that is greater than one and X is a memory factor that gives exponentially more weight to more recent samples of residual signal 370 when computing the cost function. Coefficient computer 330 updates filter 310 with the new coefficient values either by replacing the previous coefficient values or amending the previous coefficient values to make them equate with the new coefficient values. Initially, the coefficient values for filter 310 are set such that filtered reference signal 350 is zero and residue signal 370 is equal to primary signal 360. After a number of iterations, however, filtered reference signal 350 converges to a form where the weighted least square error cost function is minimized and a residual signal 370 is produced that represents best case isolation of respiration sound.

Residual signal 370 is supplied as output to respiration parameter estimator 280, which computes values for one or more respiration parameters, such as respiration rate, fractional inspiration time and/or I:E and provides the respiration parameter values to output system 130.

In some embodiments, processing system 120 performs at least some of the processing operations described herein in custom logic rather than software.

Output system 130 has a display screen for displaying respiration information determined using respiration parameter estimates received from processing system 120. In some embodiments, output system 130, in addition to a display screen, has an interface to an internal or external data management system that stores respiration information determined using respiration parameter estimates received from processing system 120 and/or an interface that transmits such information to a remote monitoring device, such as a monitoring device at a clinician facility. Respiration information outputted by output system 130 may include respiration parameter estimates received from processing system 120 and/or information derived from respiration parameter estimates, such as a numerical score or color-coded indicator of present respiratory health status.

In some embodiments, capture system 110, processing system 120 and output system 130 are part of a portable ambulatory monitoring device that monitors a person's respiratory well being in real-time as the person goes about daily activities. In other embodiments, capture system 110, processing system 120 and output system 130 may be part of separate devices that are remotely coupled via wired or wireless communication links.

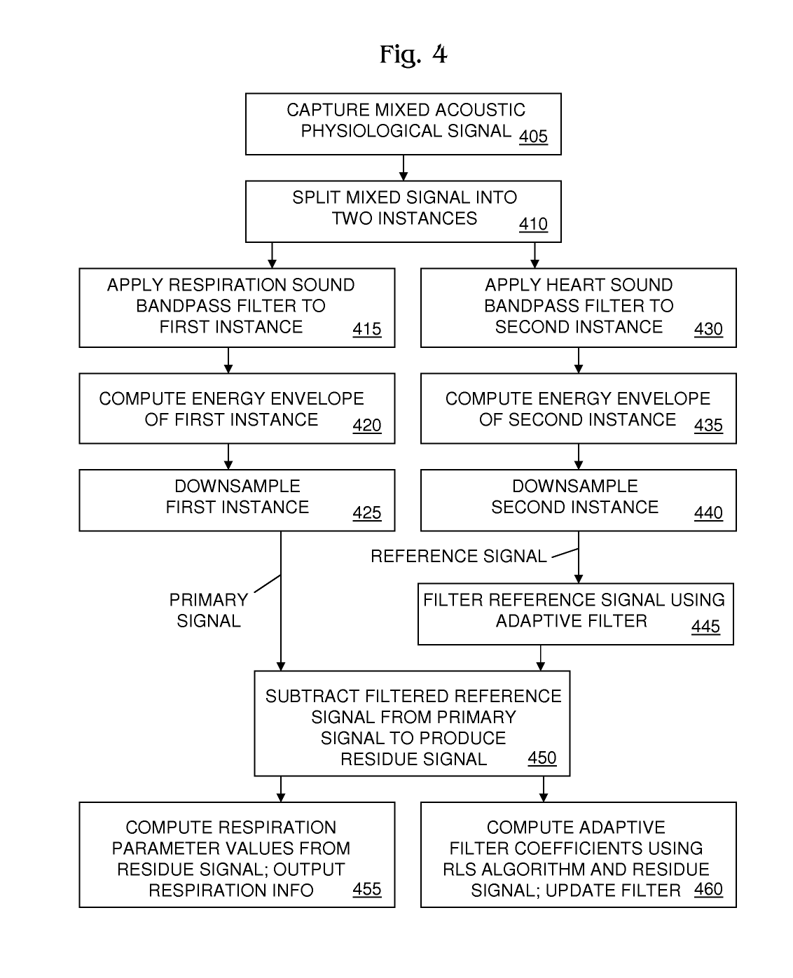

FIG. 4 shows an RLS adaptive acoustic signal filtering method performed by physiological monitoring system 100 in some embodiments of the invention. System 100 captures an acoustic physiological signal containing both respiration and heart sounds (405). System 100 splits the mixed signal into two instances (410). System 100 applies a respiration sound bandpass filter 210 to a first instance of the mixed signal (415), then computes an energy envelope of the first instance of the mixed signal (420) and then downsamples the first instance of the mixed signal (425) to generate a primary signal 360. Primary signal 360 contains both respiration sound and a meaningful level of residual heart sound. System 100 applies a heart sound bandpass filter 240 to a second instance of the mixed signal (430), then computes an energy envelope of the second instance of the mixed signal (435) and then downsamples the second instance of the mixed signal (440) to generate a reference signal 340. Reference signal 340 contains heart sound but almost no residual respiration sound. System 100 next applies adaptive filter 310 to reference signal 340 to produce filtered reference signal 350 (445) and subtracts filtered reference signal 350 from primary signal 360 to produce residue signal 370 (450). System 100 computes values for one or more respiration parameters using residue signal 370 and outputs the respiration parameter values (455). System 100 also computes values for one or more coefficients for adaptive filter 310 in accordance with an RLS algorithm using residue signal 370 and updates adaptive filter 310 with the coefficient values (460).

It will be appreciated by those of ordinary skill in the art that the invention can be embodied in other specific forms without departing from the spirit or essential character hereof. The present description is considered in all respects to be illustrative and not restrictive. The scope of the invention is indicated by the appended claims, and all changes that come with in the meaning and range of equivalents thereof are intended to be embraced therein.

What is claimed is:

1. A recursive least squares (RLS) adaptive acoustic physiological signal filtering method, comprising the steps of:
capturing by a physiological monitoring system a mixed acoustic physiological signal containing respiration sound and heart sound;
producing by the system a primary signal at least in part by applying a respiration sound bandpass filter to a first instance of the mixed signal;
producing by the system a reference signal at least in part by applying a heart sound bandpass filter to a second instance of the mixed signal;
producing by the system a filtered reference signal at least in part by applying an adaptive filter to the reference signal;
producing by the system a residue signal at least in part by subtracting the filtered reference signal from the primary signal;
computing by the system one or more values for one or more respiration parameters using the residue signal;
outputting by the system respiration information based at least in part on the respiration parameter values;
computing by the system one or more values for one or more coefficients for the adaptive filter in accordance with an RLS algorithm using the residue signal; and
updating by the system the adaptive filter with the coefficient values.

2. The method of claim 1, wherein the primary signal is further produced by computing an energy envelope of the first instance of the mixed signal.

3. The method of claim 1, wherein the primary signal is further produced by downsampling the first instance of the mixed signal.

4. The method of claim 1, wherein the reference signal is further produced by computing an energy envelope of the second instance of the mixed signal.

5. The method of claim 1, wherein the reference signal is further produced by downsampling the second instance of the mixed signal.

6. The method of claim 1, wherein the respiration sound bandpass filter and the heart sound bandpass filter have respective passbands that partially overlap.

7. The method of claim 1, wherein the respiration sound bandpass filter has a passband from 80 Hz plus or minus ten percent to 300 Hz plus or minus ten percent.

8. The method of claim 1, wherein the heart sound bandpass filter has a high cutoff frequency from 10 Hz plus or minus ten percent to 100 Hz plus or minus ten percent.

9. The method of claim 1, further comprising the step of splitting by the system the mixed signal into the first instance and the second instance.

10. The method of claim 1, further comprising the step of amplifying by the system the mixed signal.

11. The method of claim 1, further comprising the step of applying by the system a lowpass filter to the mixed signal.

12. The method of claim 1, wherein the respiration parameters include respiration rate.

13. The method of claim 1, wherein the system is an ambulatory monitoring system.

14. A physiological monitoring system, comprising:
a sound capture system configured to capture a mixed acoustic physiological signal containing respiration sound and heart sound;
an acoustic signal processing system operatively coupled with the capture system and configured to produce a primary signal at least in part by applying a respiration sound bandpass filter to a first instance of the mixed signal, produce a reference signal at least in part by applying a heart sound bandpass filter to a second instance of the mixed signal, produce a filtered reference signal at least in part by applying an adaptive filter to the reference signal, produce a residue signal at least in part by subtracting the filtered reference signal from the primary signal, compute one or more values for one or more respiration parameters using the residue signal, output the respiration parameter values, compute one or more values for one or more coefficients for the adaptive filter in accordance with a recursive least squares (RLS) algorithm using the residue signal and update the adaptive filter with the coefficient values; and
a physiological data output system operatively coupled with the processing system and configured to output respiration information based at least in part on the respiration parameter values.

15. The system of claim 14, wherein the primary signal is further produced by computing an energy envelope of the first instance of the mixed signal.

16. The system of claim 14, wherein the reference signal is further produced by computing an energy envelope of the second instance of the mixed signal.

17. The system of claim 14, wherein the system is an ambulatory monitoring system.
